(19) United States
(12) Patent Application Publication
(10) Pub. No.: US 2018/0358123 A1
(43) Pub. Date: Dec. 13, 2018

(54) INTERACTIVE SCHEDULING WEB PORTAL

(71) Applicant: Matthew Silver, Virginia Beach, VA (US)

(72) Inventor: Matthew Silver, Virginia Beach, VA (US)

(21) Appl. No.: 16/004,360

(22) Filed: Jun. 9, 2018

Related U.S. Application Data
(60) Provisional application No. 62/517,816, filed on Jun. 9, 2017.

(51) Int. Cl. G16H 40/20; G06Q 10/10; G16H 10/60; G06F 17/30

(52) U.S. Cl. CPC G16H 40/20; G06F 17/30289; G16H 10/60; G06Q 10/1095

(57) ABSTRACT

A scheduling portal including a processor, a memory on which are stored machine readable instructions that when executed by the processor, cause the processor to: access medical provider data for a plurality of medical providers and store the medical provider data in a database, receive patient parameters from a patient mobile device and store the patient parameters in the database, determine if the patient parameters contain a scheduling request for at least one of the medical providers having the medical provider data stored in the database, schedule a requested appointment for the patient with the medical provider if the patient parameters contain a scheduling request, update a medical provider calendar in the database, and send the updated medical provider calendar to a medical provider mobile device.

### FIELD OF INVENTION

The present general inventive concept relates to a system and a method of providing a secure communication portal between home health care providers (i.e., first users), patients (i.e., second users), and other third parties (i.e., third users) to allow for interactive scheduling of appointments. The present general inventive concept also relates to a secure communication portal that displays interactive electronic schedules allowing first users to navigate to second users locations and indicate completion of appointments.

### BACKGROUND

In home health care, patients often become upset when a home health care provider is late or cancels an appointment at the last moment. Patients want to know precise arrival times of the health care provider so that they can schedule other activities, such as work, before and/or after the scheduled appointment. As a result, appointment delays cost the patients and/or their family members a significant of amount of money when missing work in order to be available for the scheduled appointments.

In addition, there is an excess of paper documents that need to be securely stored which occupies a considerable amount of space.

Therefore, what is needed is an interactive communication portal that allows home health care patients to know the exact arrival time of their health care provider, in real-time. Also, the interactive communication portal should be easily accessible as a web-based application and allow communication between the home health care provider, the patient, and other third parties.

In addition, what is also needed is a completely electronic and paperless system which provides health care providers seamless access to the patient's health records in a secure and HIPAA compliant manner and which allows for electronic signature verification.

### BRIEF SUMMARY OF THE INVENTION

According to one aspect of the present general inventive concept, the present invention provides a scheduling portal including a processor, a memory on which are stored machine readable instructions that when executed by the processor, cause the processor to access medical provider data for a plurality of medical providers and store the medical provider data in a database, receive patient parameters from a patient mobile device and store the patient parameters in the database, determine if the patient parameters contain a scheduling request for at least one of the medical providers having the medical provider data stored in the database, schedule a requested appointment for the patient with the medical provider if the patient parameters contain a scheduling request, update a medical provider calendar in the database, and send the updated medical provider calendar to a medical provider mobile device.

The instructions may further cause the processor to check medical provider availability for the requested appointment.

The instructions may further cause the processor to send a notification to the patient mobile device if the medical provider is not available.

The instructions may further cause the processor to send a notification to the patient mobile device if the medical provider is available, wherein the notification renders the updated medical provider calendar on the patient mobile device.

The instructions may further cause the processor to track medical provider movement and update the medical provider calendar.

The instructions may further cause the processor to send the updated medical provider calendar to the patient mobile device to render the updated medical provider calendar to the patient.

The instructions may further cause the processor to send and render a map depicting locations of appointments to the medical provider mobile device.

The database may be a local database.

The database may be a cloud storage.

According to another aspect of the present general inventive concept, the present invention provides a method for scheduling appointments using a scheduling portal, the method includes accessing, by a processor of the scheduling portal, medical provider data for a plurality of medical providers and store the medical provider data in a database, receiving, by the processor of the scheduling portal, patient parameters from a patient mobile device and storing the patient parameters in the database, determining, by the processor of the scheduling portal, if the patient parameters contain a scheduling request for at least one of the medical providers having the medical provider data stored in the database, scheduling, by the processor of the scheduling portal, a requested appointment for the patient with the medical provider if the patient parameters contain a scheduling request, updating, by the processor of the scheduling portal, a medical provider calendar in the database, and sending, by the processor of the scheduling portal, the updated medical provider calendar to a medical provider mobile device.

The method may further include checking medical provider availability for the requested appointment.

The method may further include sending a notification to the patient mobile device if the medical provider is available, wherein the notification renders the updated medical provider calendar on the patient mobile device.

The method may further include tracking medical provider movement and updating the medical provider calendar based on a GPS location retrieved from the medical provider mobile device.

The method may further include sending the updated medical provider calendar to the patient mobile device to render the updated medical provider calendar to the patient.

The method may further include sending a map depicting locations of appointments to the medical provider mobile device to be rendered to the medical provider.

According to another aspect of the present general inventive concept, the present invention provides an automated scheduling portal including a processor, a memory on which are stored machine readable instructions that when executed by the processor, cause the processor to receive patient parameters from a patient mobile device and store the patient parameters in a database, retrieve pre-stored medical provider data from the database based on the patient parameters, send the medical provider data to the patient mobile device, receive a scheduling request for the medical provider from the patient mobile device, schedule an appointment for the patient with the medical provider, update a medical provider calendar in the database, and send the updated medical provider calendar to a medical provider mobile device.

The instructions may further cause the processor to derive a patient family member mobile device data from the patient parameters.

The instructions may further cause the processor to provide the appointment data to the patient family member mobile device.

The instructions may further cause the processor to track medical provider movement and update the medical provider calendar based on a GPS location retrieved from the medical provider mobile device.

The instructions may further cause the processor to send a map depicting a location of medical provider appointments to the patient family member mobile device to be rendered to the patient family member.

Additional aspects of the present general inventive concept will be set forth in part in the description which follows and, in part, will be obvious from the description, or may be learned by practice of the general inventive concept.

### BRIEF DESCRIPTION OF THE DRAWINGS

These and/or other aspects of the present general inventive concept will become apparent and more readily appreciated from the following description of the embodiments, taken in conjunction with the accompanying drawings of which:

FIG. 1 is a schematic block diagram of a system having interactive scheduling web portal instructions stored on a computer readable medium, according to the present general inventive concept;

FIG. 2 is a schematic diagram illustrating an operation of the interactive scheduling web portal application system in FIG. 1;

FIG. 3A is a diagram of an interactive scheduling web portal system, according to the present general inventive concept;

FIG. 3B is a diagram of modules within the interactive scheduling web portal system illustrated in FIG. 3A, according to the present general inventive concept;

FIG. 4 is flow chart of a method executed by the interactive scheduling web portal, according to the present general inventive concept;

FIG. 5 is flow chart of a method executed by the interactive scheduling web portal for rendering data to users, according to the present general inventive concept;

FIG. 6 illustrates an example computer system/server residing on a computing node configured to support one or more of the example embodiments;

FIGS. 7 through 18 are exemplary embodiments of screen shots displayed on a display screen, implementing the interactive scheduling web portal system depicted in FIGS. 3A and 3B;

FIG. 19 illustrates a block diagram of an example portable computing device implementing the interactive scheduling web portal system, according to the present general inventive concept; and

FIG. 20 illustrates a block diagram of another example embodiment of a portable computing device used to implement the interactive scheduling web portal system, according to the present general inventive concept.

### DETAILED DESCRIPTION OF THE INVENTION

Reference will now be made in detail to the exemplary embodiments of the present general inventive concept, examples of which are illustrated in the accompanying drawings, wherein like reference numerals refer to the like elements throughout. The exemplary embodiments are described below in order to explain the present general inventive concept by referring to the figures.

The present general inventive concept provides a system utilizing a software application which is used to execute a method for scheduling appointments remotely via mobile devices.

The present general inventive concept provides an interactive scheduling web portal system utilizing a software application which is used to execute a method to increase an efficiency (e.g., time or cost) of scheduling home health care visits or appointments of health care providers with patients by allowing health care providers, patients, and other third parties (i.e., family of patients) to view real-time updates on arrival time of the health care provider to the predefined appointment location.

The present general inventive concept provides an interactive scheduling web portal system accessible from a smart phone, tablet, or other mobile device. The interactive scheduling web portal system and method allows a health care provider to quickly visualize all of his or her scheduled appointments with patients on a single screen and allows the health care provider to press a single button to display a map and directions to the appointment location (e.g., patient's home). In addition, the interactive scheduling web portal system and method allows the health care provider to press a single button to record within a central database that the appointment has been completed.

The present general inventive concept also provides an interactive scheduling web portal application or program that provides patients with a two to four-hour window of arrival time of the health care provider and provides patient with access to view real-time changes in arrival time.

The present general inventive concept also provides an interactive scheduling web portal application or program that provides a secure and efficient data collection and retrieval system to manage the patient records.

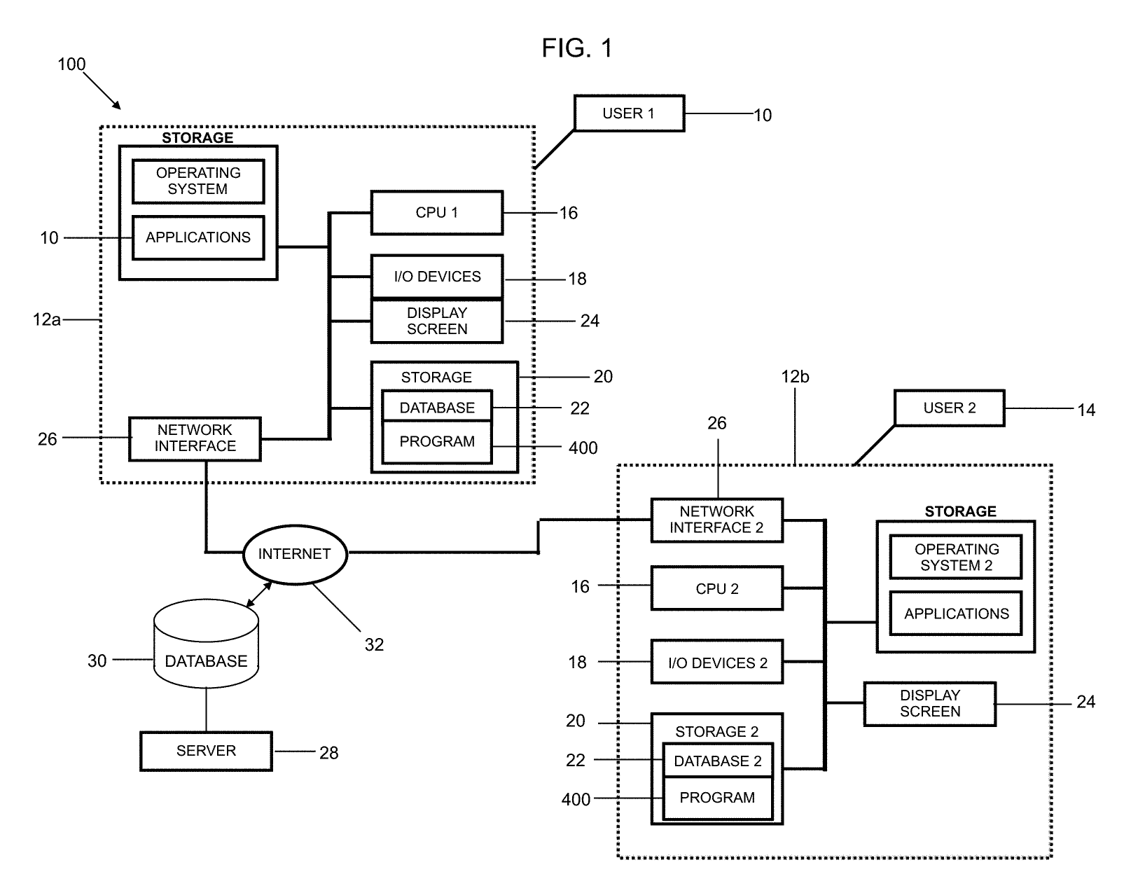

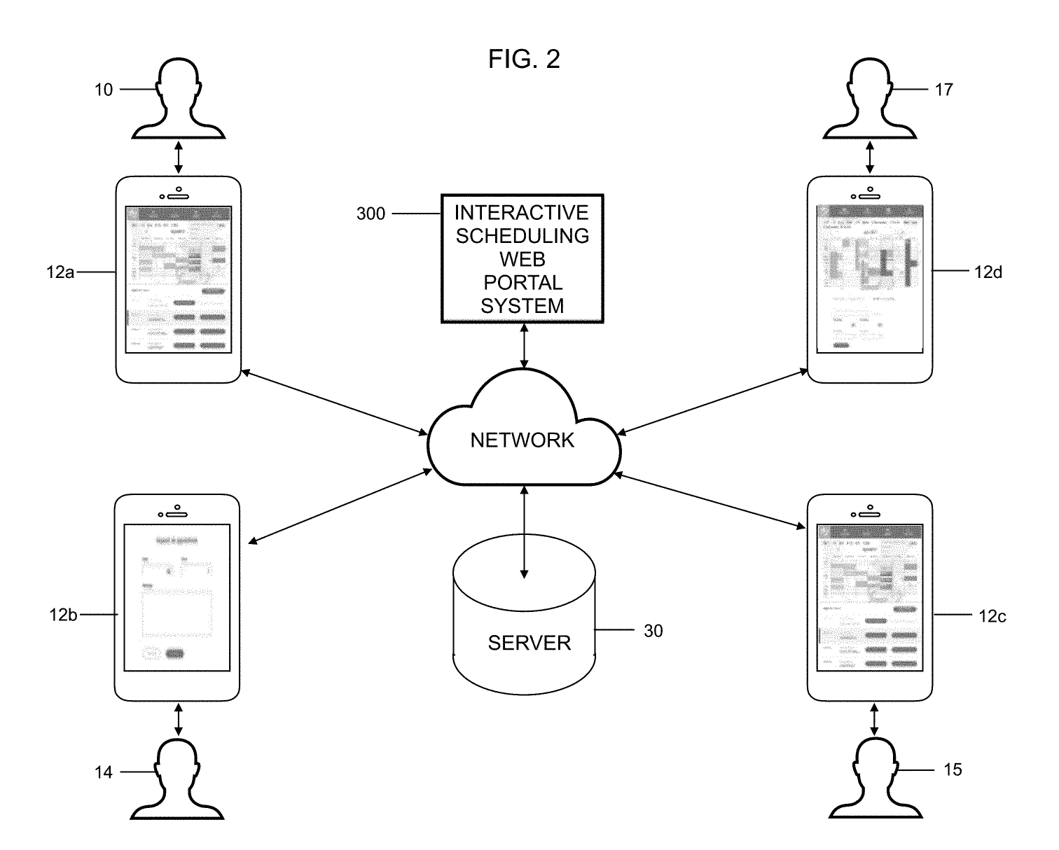

FIG. 1 is a schematic block diagram of a system 100 having interactive scheduling web portal instructions stored on a computer readable medium, according to the present general inventive concept. FIG. 2 is a schematic diagram illustrating an operation of the interactive scheduling web portal application system in FIG. 1.

Referring to FIGS. 1 and 2, the system 100 according to the present general inventive concept provides a plurality of users (i.e., a first user, a second user, a third user, and an admin user) with access to an interactive scheduling web portal 300 that receives and stores user profile information and maintains home health care scheduling between the first user (i.e., clinician) and the second user (i.e., patient).

In the present embodiment, a first user 10 may use a first portable computing device 12a to access, view, and/or modify scheduling information within a schedule module of the interactive scheduling web portal 300. In addition, the first user 10 (e.g., health care provider or clinician) may use a first portable computing device 12a (i.e., first mobile device) to communicate various information including appointment scheduling information directly with a second user 14 (e.g., health care patient) using a second portable computing device 12b (i.e., first mobile device) or with a third user 15 (e.g., family member of the patient) using a third portable computing device 12c (i.e., mobile device). In the present embodiment, only a fourth user 17 (i.e., admin user) using a fourth computing device 12d may view, edit, modify the profile, appointment, and schedule information of all first, second, and third users registered and stored within the interactive scheduling web portal 300.

In exemplary embodiments, each user (10, 14, 15, 17) may use a portable computing device 12a, 12b, 12c (e.g., a mobile device or a tablet) which may include a CPU 16, input and output devices 18, storage 20 to store instructions 400 according to the present invention within a database 22, a display screen 24, a network interface 26 to communicate with an external server 28 and other databases 30 or mobile devices via an internet network 32.

In alternative embodiments, the portable computing device may further include an additional storage to store and execute various other features of the modules within the interactive scheduling web portal system 100.

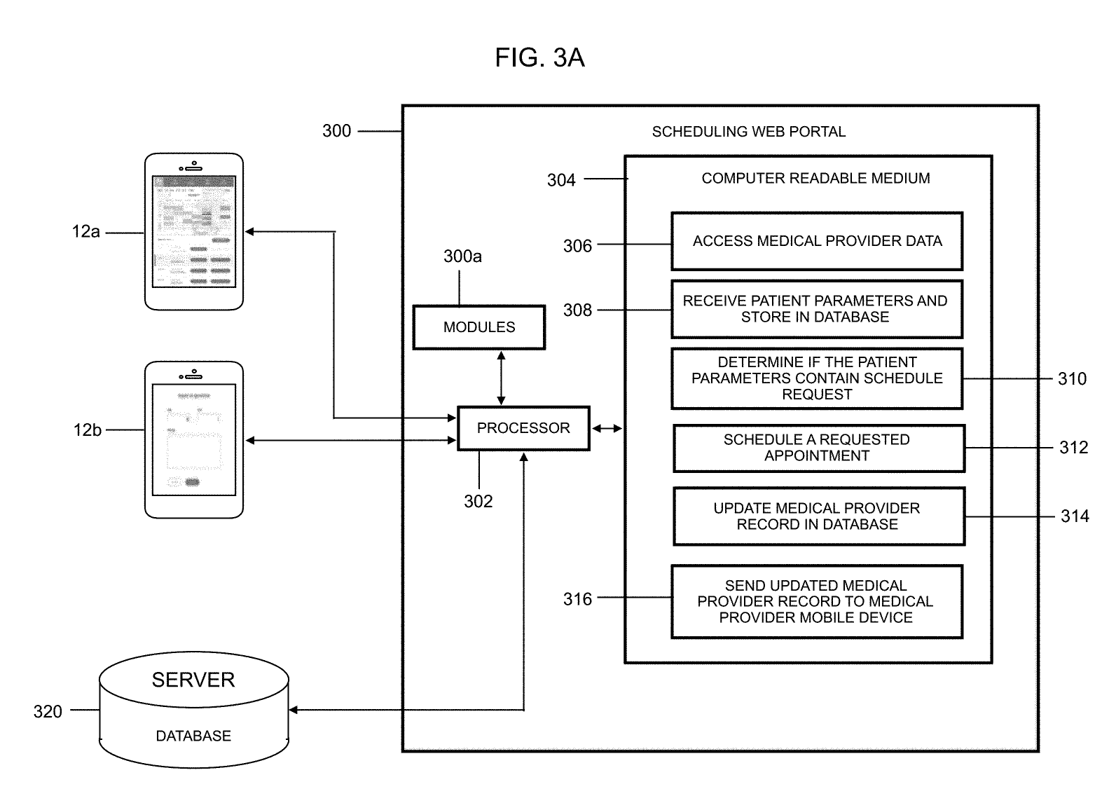

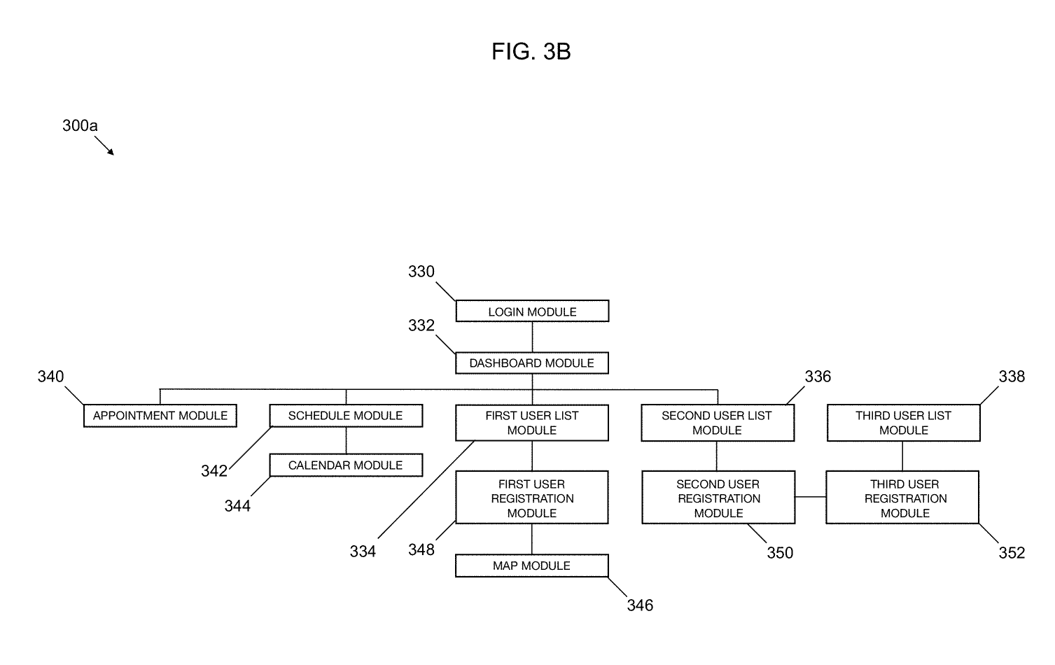

FIG. 3A illustrates a diagram of an interactive scheduling web portal system 300, according to the present general inventive concept. It should be understood that the scheduling web portal 300 may include additional components and that some of the components described herein may be removed and/or modified without departing from a scope of the web portal 300 disclosed herein.

Referring to FIG. 3A, in the present embodiment, the interactive scheduling web portal 300 may be a computing device, a tablet computer, a server computer, a Smartphone, or the like, and may include a processor 302, which may be a semiconductor-based microprocessor, a central processing unit (CPU), an application specific integrated circuit (ASIC), a field-programmable gate array (FPGA), and/or another hardware device. Although a single processor 302 is depicted, it should be understood that the web portal system 300 may include multiple processors, multiple cores, or the like, without departing from a scope of the web portal system 300.

The web portal system 300 may also include a non-transitory computer readable medium 304 that may have stored thereon machine-readable instructions executable by the processor 302. Examples of the machine-readable instructions are shown as 306-316 and are further discussed below. Examples of the non-transitory computer readable medium 304 may include an electronic, magnetic, optical, or other physical storage device that contains or stores executable instructions. For example, the non-transitory computer readable medium 304 may be a Random-Access memory (RAM), an Electrically Erasable Programmable Read-Only Memory (EEPROM), a hard disk, an optical disc, or other type of storage device.

The processor 302 may fetch, decode, and execute the machine-readable instructions 306 to access medical provider data for a plurality of medical providers and store the medical provider data in a database 320. In other words, the medical providers' data may be pre-stored in the database 320, which may be a local database or a distributed database such as a cloud storage. The processor 302 may fetch, decode, and execute the machine-readable instructions 308 to receive patient parameters from the first user device 12a, the second user device 12b and store the patient parameters in the database 320. The patient parameters may include patient's age, diagnosis, address etc. For a new patient the registration data may be used. The patient parameters may also include an appointment request with a particular medical provider or with a certain type of medical providers.

The processor 302 may fetch, decode, and execute the machine-readable instructions 310 to determine if the patient parameters contain a scheduling request for at least one of the medical providers having the medical provider data stored in the database 320. The processor 302 may fetch, decode, and execute the machine-readable instructions 312 to schedule a requested appointment for the patient with the medical provider if the patient parameters contain a scheduling request. The processor 302 may fetch, decode, and execute the machine-readable instructions 314 to update a medical provider calendar in the database 320. The processor 302 may fetch, decode, and execute the machine-readable instructions 316 to send the updated medical provider calendar to a medical provider mobile device 12a.

FIG. 3B is a diagram of modules 300a within the interactive scheduling web portal system 300 illustrated in FIG. 3A, according to the present general inventive concept.

Referring to FIG. 3B, in exemplary embodiments, the interactive scheduling web portal system 300 includes a login module 330 configured to authenticate a user, a dashboard module 332 configured to generate and display items accessible to each user based on their profile information, a first user list module 334 configured to generate and display a list of all first users (i.e., clinicians or health providers), a second user list module 336 configured to generate and display a list of all second users (i.e., patients), a third user list module 338 configured to generate and display a list of all third users (i.e., family members), an appointment module 340 configured to receive appointment input information, a schedule module 342 configured to generate a schedule of all scheduled appointments, a calendar module 344 configured to overlay scheduled appointments on a calendar, and a map module 346 to superimpose a location of scheduled appointments on a map image. In addition, the web portal system 300 may further include a first user registration module 348 to register a first user, a second user registration module 350 to register a second user, and a third user registration module 352 to register a third user.

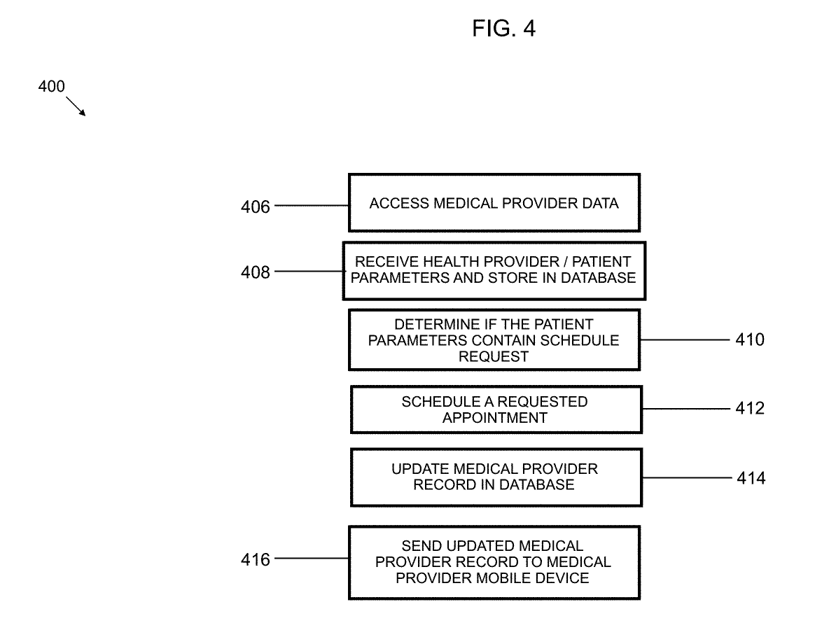

FIG. 4 illustrates a flow chart of a scheduling method executed by the interactive scheduling web portal 300, according to the present general inventive concept. It should be understood that method 400 depicted in FIG. 4 may include additional operations and that some of the operations described therein may be removed and/or modified without departing from the scope of the method 400. The description of the method 400 is also made with reference to the features depicted in FIG. 3A for purposes of illustration. Particularly, the processor 302 of the scheduling portal 300 may execute some or all of the operations included in the method 400.

With reference to FIG. 4, at block 406, the processor 302 may access medical provider data for a plurality of medical providers and store the medical provider data in a database 320. At block 408, the processor 302 may receive first user (i.e., health care provider) parameters and/or second user (i.e., patient) parameters from a patient mobile device and store the patient parameters in the database 320 using the first or second user list module 334, 336. At block 410, the processor 302 may determine if the patient parameters contain a scheduling request for at least one of the medical providers having the medical provider data stored in the database 320. At block 412, the processor 302 may schedule a requested appointment for the patient with the medical provider if the patient parameters contain a scheduling request. At block 414, the processor 302 may update a medical provider calendar in the database 320. Then, at block 416, the processor 302 may send the updated medical provider calendar to a first user or medical provider mobile device 12a.

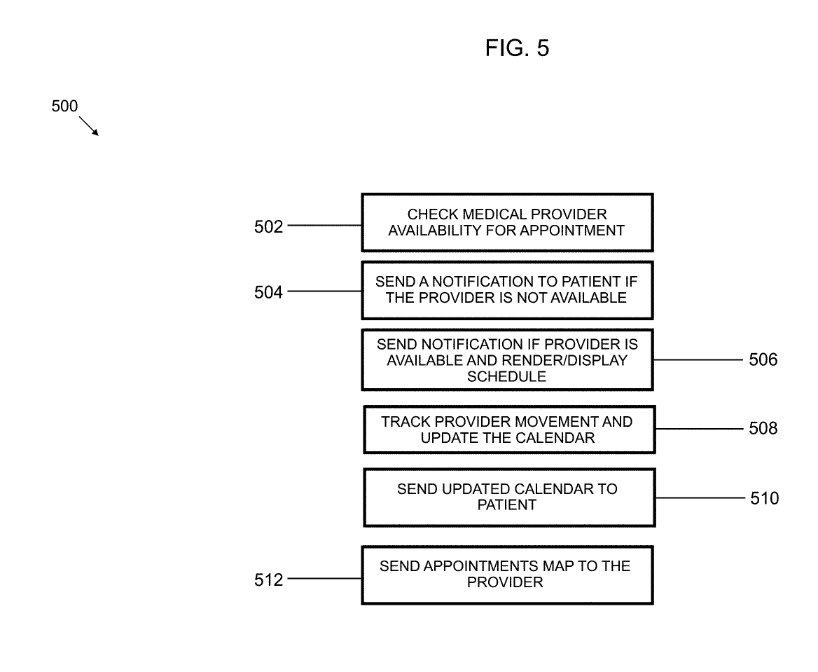

FIG. 5 illustrates a flow chart of a scheduling method executed by the interactive scheduling web portal 300 and rendering the schedule-related data to multiple parties including patients and medical providers, according to the present general inventive concept. It should be understood that method 500 depicted in FIG. 5 may include additional operations and that some of the operations described therein may be removed and/or modified without departing from the scope of the method 500. The description of the method 500 is also made with reference to the features depicted in FIG. 3A for purposes of illustration. Particularly, the processor 302 of the scheduling portal 300 may execute some or all of the operations included in the method 500.

With reference to FIG. 5, at block 502, the processor 302 may check medical provider availability for the requested appointment. For example, the processor can may retrieve a schedule file from the database and parse it to determine if the requested time and date is available. At block 504, the processor 302 may send a notification to the patient mobile device if the medical provider is not available. At block 506, the processor 302 may send a notification to the patient mobile device if the medical provider is available. The notification may render the updated medical provider calendar on the patient mobile device so the user can visually see his or her appointment on the provider's calendar. At block 508, the processor 302 may track medical provider movement and update the medical provider calendar. For example, if the GPS indicates that the provider is not going to be on time, the provider's calendar can be updated so the patients can see the actual provider arrival times. At block 510, the processor 302 may send the updated medical provider calendar to the patient mobile device to render the updated medical provider calendar to the patient. At block 512, the processor 302 may send and render a map M1 depicting locations of appointments A1 to the medical provider mobile device so that the medical provider can see his route and optimize his traveling time if needed.

Through implementation of the methods 400 and 500, the scheduling portal 300 may be able to automatically schedule and re-schedule patient appointments with the medical provider. Some or all of the operations set forth in the methods 400 and 500 may be contained as utilities, programs, or subprograms, in any desired computer accessible medium. In addition, the method 400 and 500 may be embodied by computer programs, which may exist in a variety of forms. For example, the methods 400 and 500 may exist as machine readable instructions, including source code, object code, executable code or other formats. Any of the above may be embodied on a non-transitory computer readable storage medium.

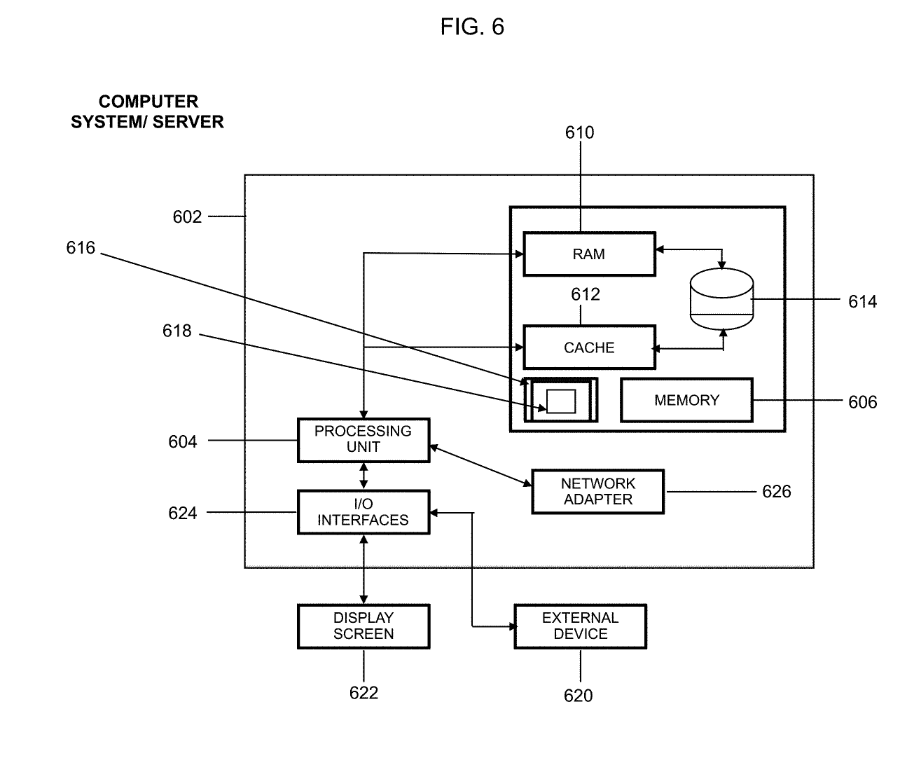

FIG. 6 illustrates an example computer system/server residing on a computing node configured to support one or more of the example embodiments.

FIG. 6 illustrates an example computing node hosting a computer system/server configured to support one or more of the example embodiments. FIG. 6 is not intended to suggest any limitation as to the scope of use or functionality of embodiments of the application described herein. Regardless, the computing node is capable of being implemented and/or performing any of the functionality set forth hereinabove.

In computing node there is a computer system/server 602, which is operational with numerous other general purpose or special purpose computing system environments or configurations. Examples of well-known computing systems, environments, and/or configurations that may be suitable for use with computer system/server 602 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 602 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 602 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 602 residing on a computing node is shown in the form of a general-purpose computing device. The components of computer system/server 602 may include, but are not limited to, one or more processors or processing units 604, a system memory 606, and a bus that couples various system components including system memory 606 to processor 604.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 602 typically includes a variety of computer system readable media. Such media may be any available media that is accessible by computer system/server 602, and it includes both volatile and non-volatile media, removable and non-removable media. System memory 606, in one embodiment, implements the flow diagrams of the other figures. The system memory 606 can include computer system readable media in the form of volatile memory, such as random-access memory (RAM) 610 and/or cache memory 612. Computer system/server 602 may further include other removable/non-removable, volatile/non-volatile computer system storage media. By way of example only, storage system 614 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory 606 may include at least one program product having a set (e.g., at least one) of program modules that are configured to carry out the functions of various embodiments of the application.

Program/utility 616, having a set (at least one) of program modules 618, may be stored in memory 606 by way of example, and not limitation, as well as an operating system, one or more application programs, other program modules, and program data. Each of the operating system, one or more application programs, other program modules, and program data or some combination thereof, may include an implementation of a networking environment. Program modules 618 generally carry out the functions and/or methodologies of various embodiments of the application as described herein.

As will be appreciated by one skilled in the art, aspects of the present application may be embodied as a system, method, or computer program product. Accordingly, aspects of the present application may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

Computer system/server 602 may also communicate with one or more external devices 620 such as a keyboard, a pointing device, a display 622, etc.; one or more devices that enable a user to interact with computer system/server 602; and/or any devices (e.g., network card, modem, etc.) that enable computer system/server 602 to communicate with one or more other computing devices. Such communication can occur via I/O interfaces 624. Still yet, computer system/server 602 can communicate with one or more networks such as a local area network (LAN), a general wide area network (WAN), and/or a public network (e.g., the Internet) via network adapter 626. As depicted, network adapter 626 communicates with the other components of computer system/server 602 via a bus. It should be understood that although not shown, other hardware and/or software components could be used in conjunction with computer system/server 602. Examples, include, but are not limited to: microcode, device drivers, redundant processing units, external disk drive arrays, RAID systems, tape drives, and data archival storage systems, etc.

Although an exemplary embodiment of at least one of a system, method, and non-transitory computer readable medium has been illustrated in the accompanied drawings and described in the foregoing detailed description, it will be understood that the application is not limited to the embodiments disclosed, but is capable of numerous rearrangements, modifications, and substitutions as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a Smartphone or any other suitable computing device, or combination of devices. Presenting the above-described functions as being performed by a “system” is not intended to limit the scope of the present application in any way but is intended to provide one example of many embodiments. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may custom physical hardware components designed and configured to function as required herein and may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

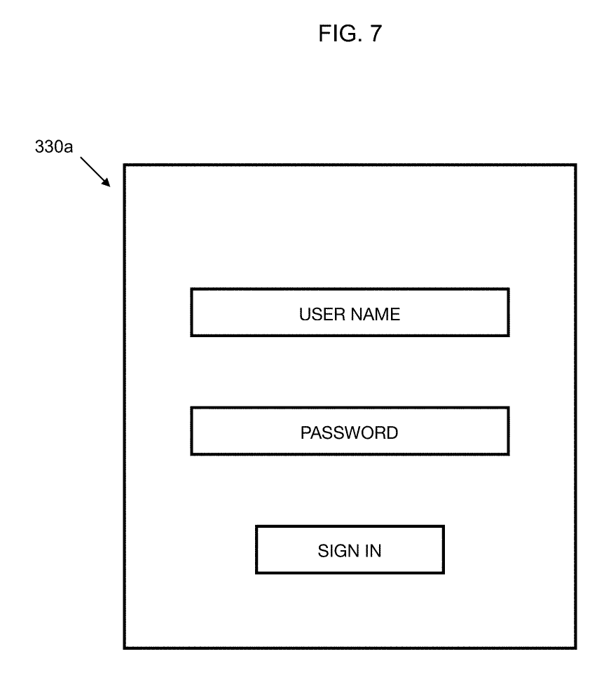

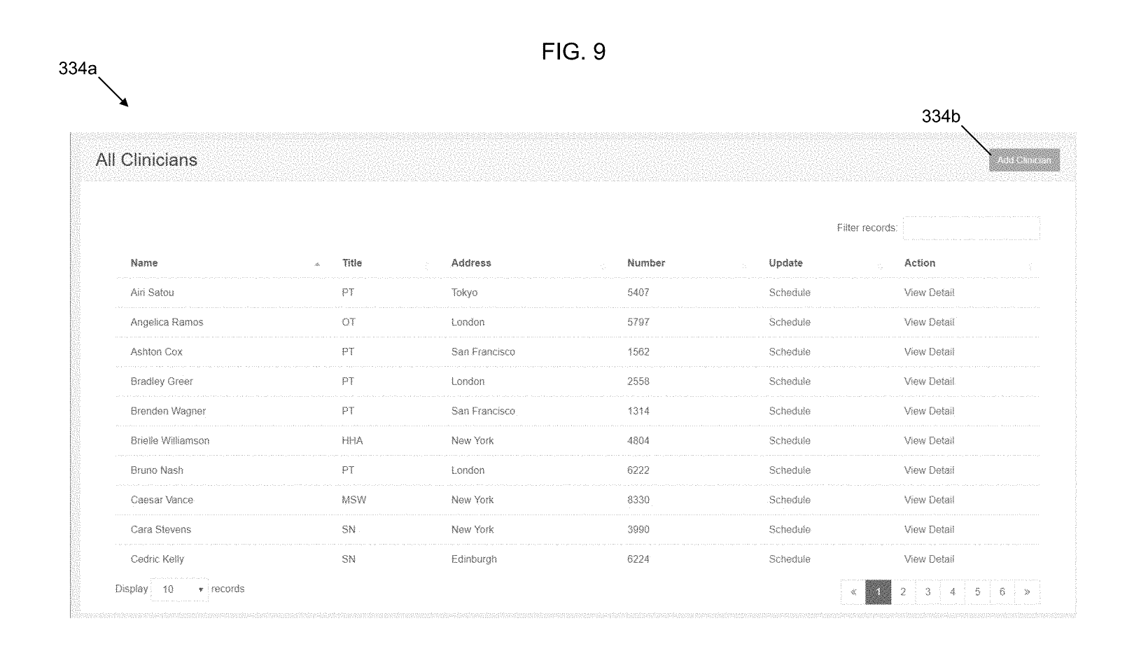

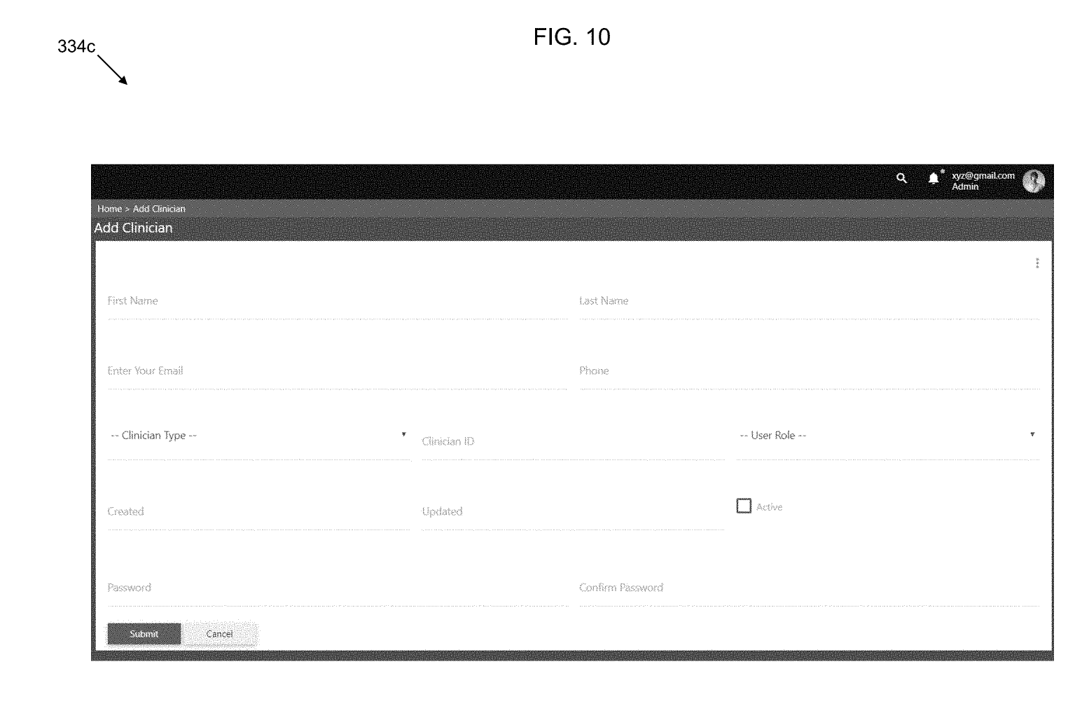

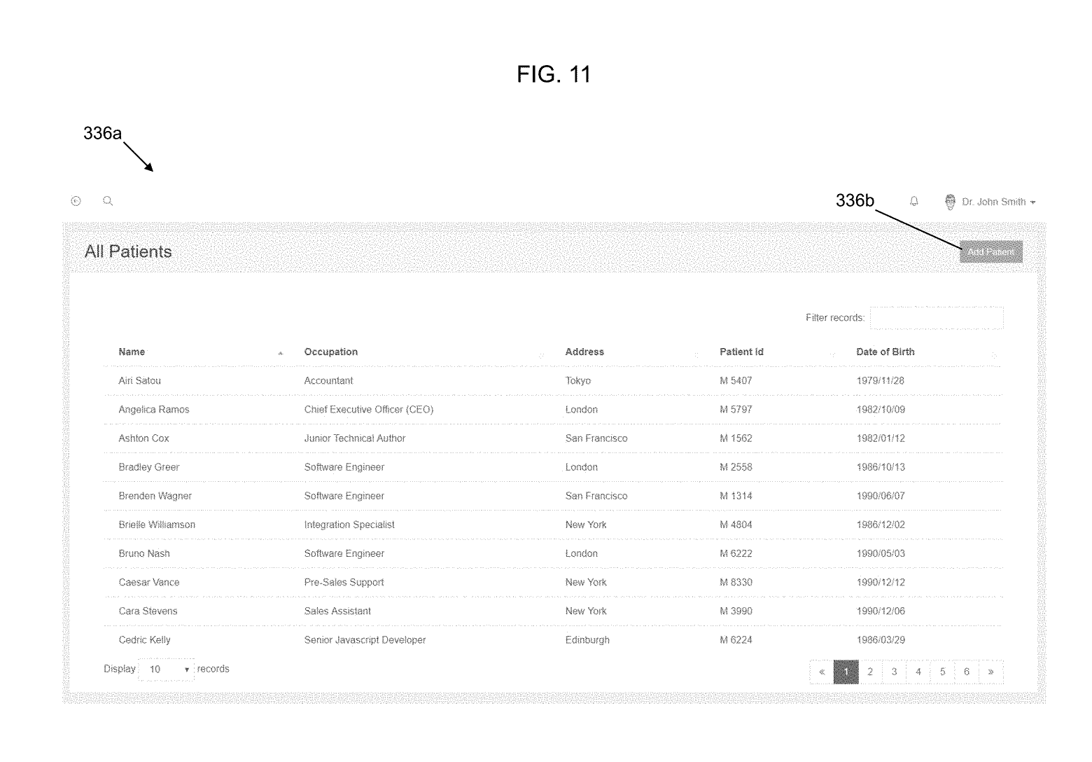

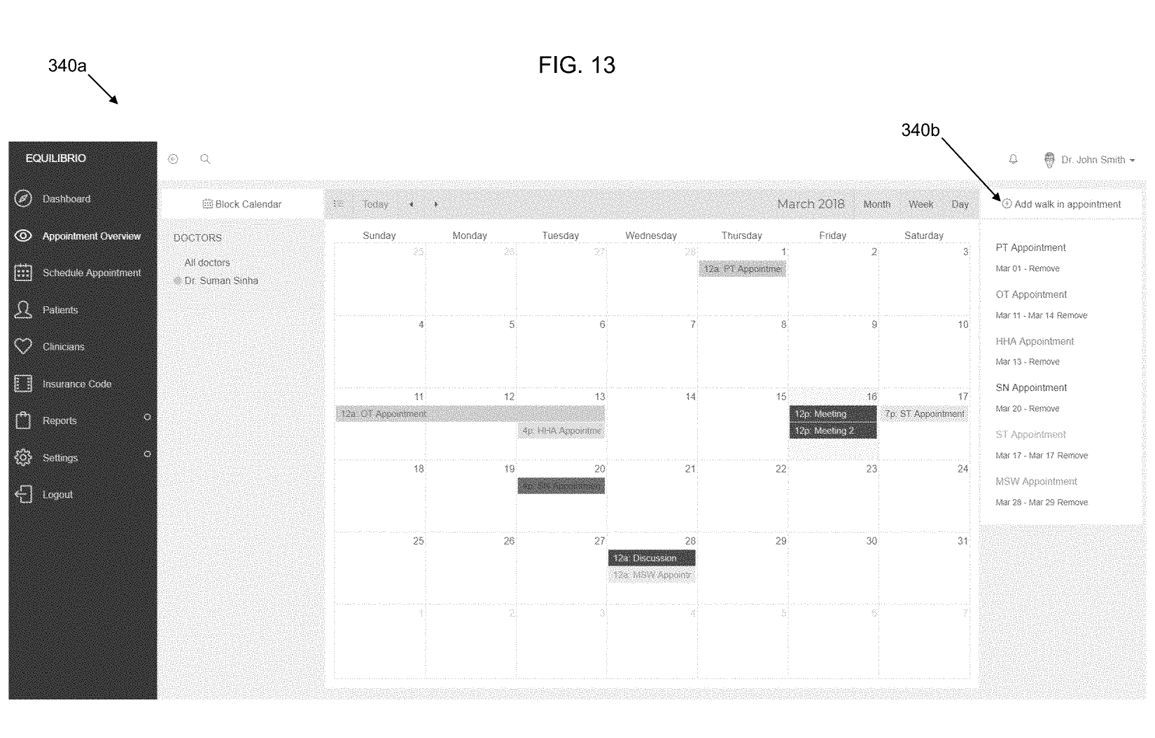

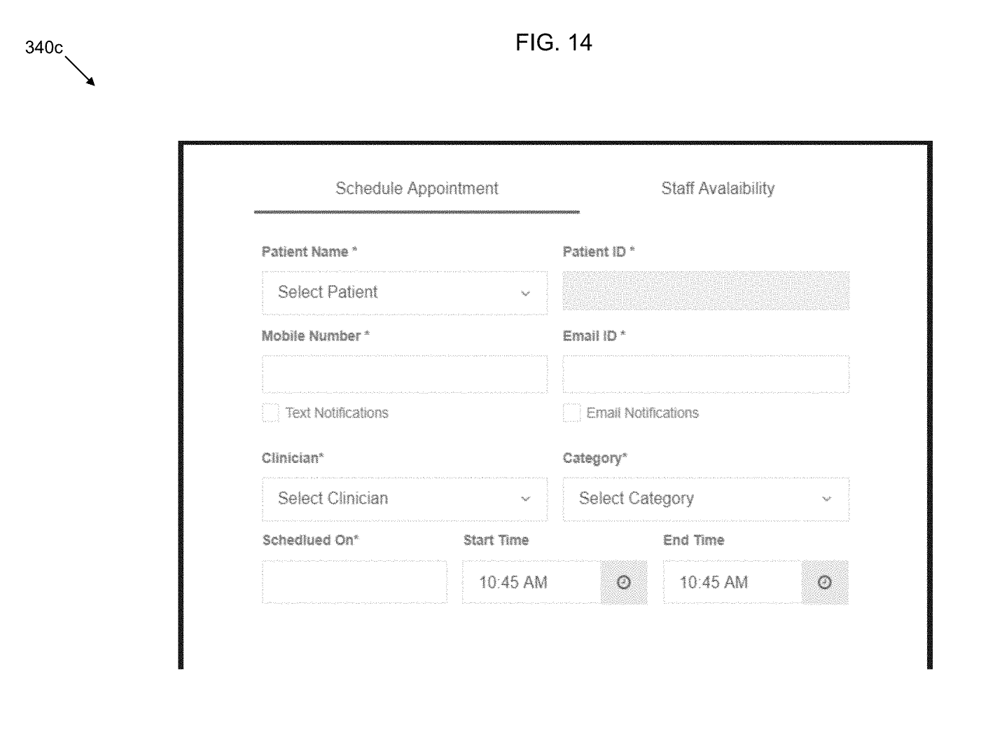

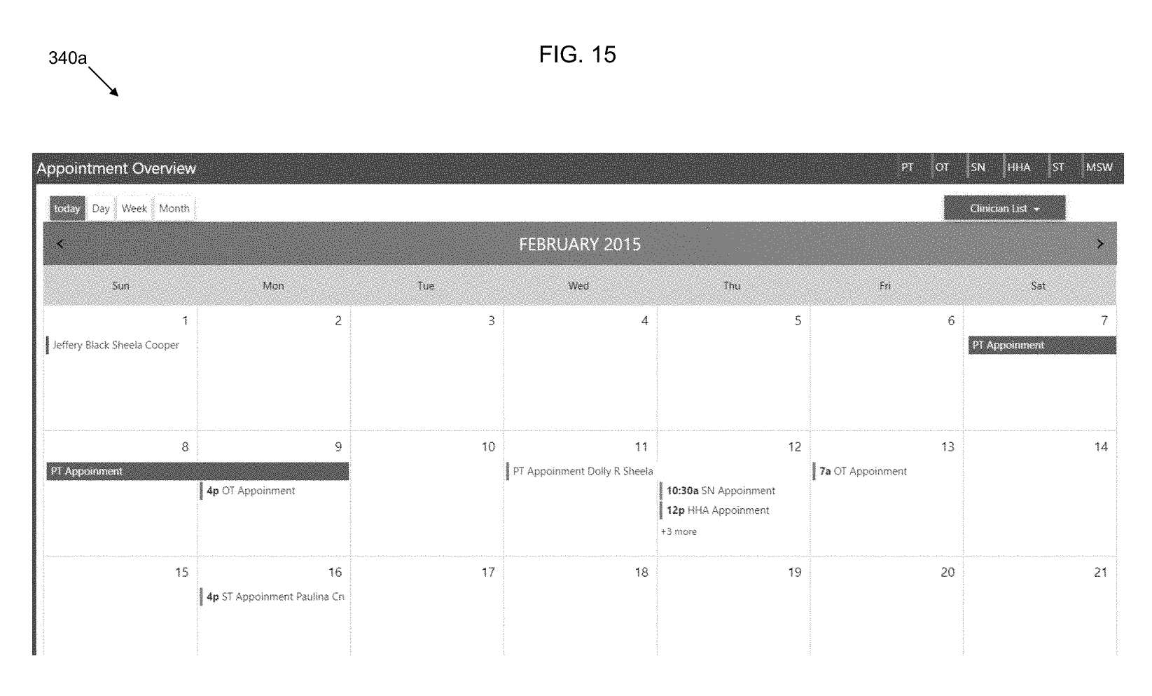

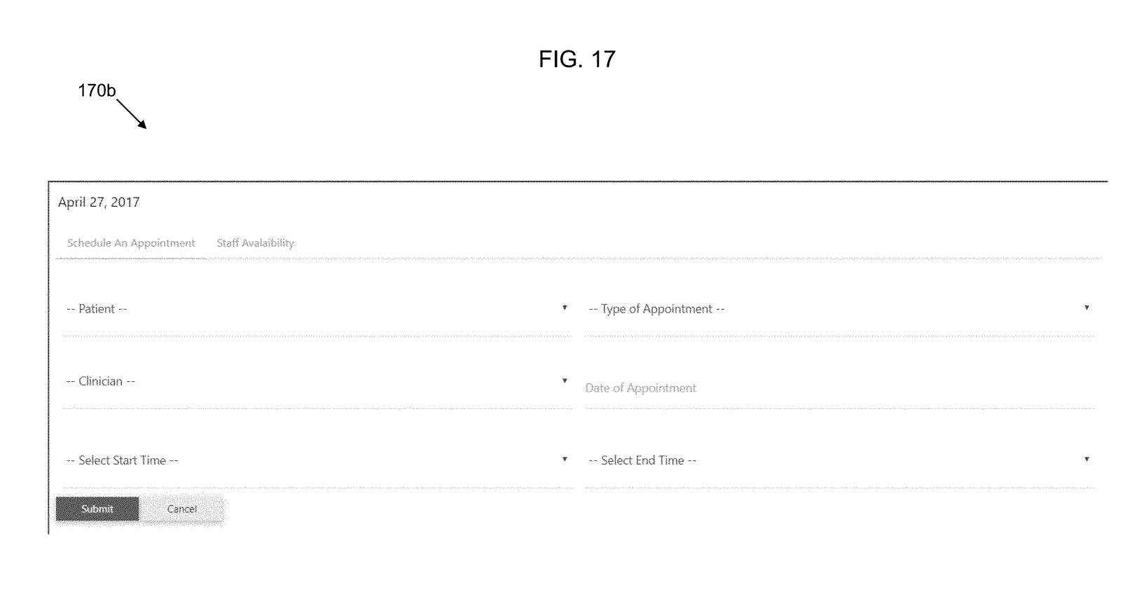

FIGS. 7 through 18 are exemplary embodiments of schematic screen shots displayed on a display screen, implementing the interactive scheduling web portal system depicted in FIGS. 3A and 3B according to the present general inventive concept.

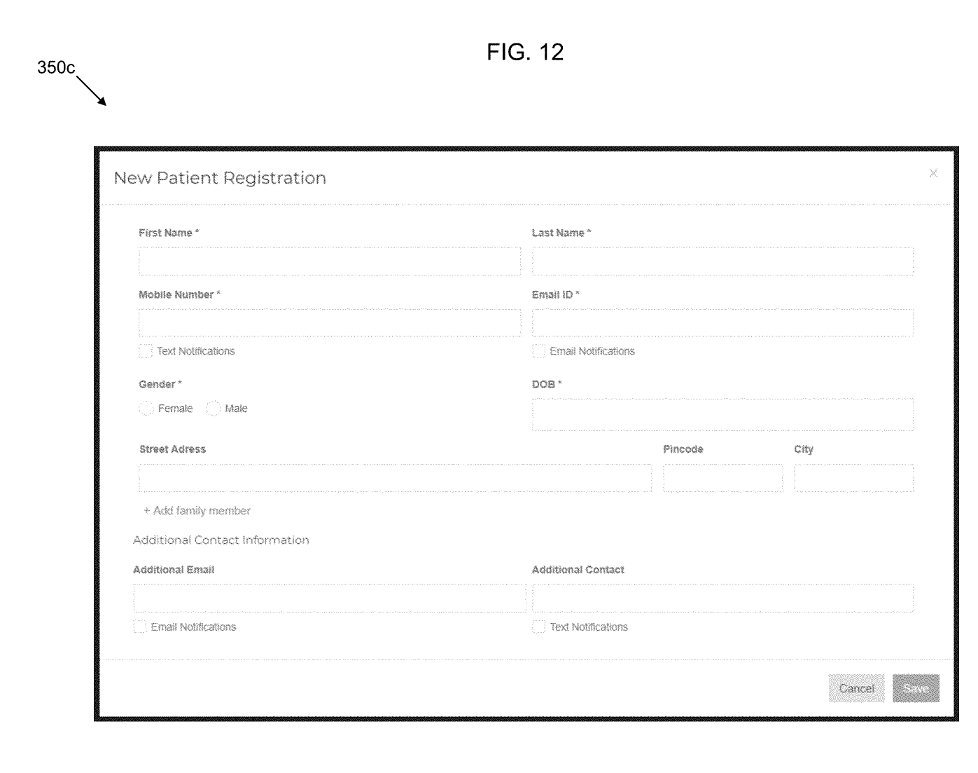

In use, referring to FIGS. 3A, 3B and 7 through 18, the interactive scheduling web portal 300 includes a login module 330 which allows the system 100 to authenticate each user attempting to access the web portal system 100, either directly or remotely. Each user must initially create, register and store a user profile with login credentials within the system 100 via a registration module. For instance, a health care provider (i.e., a first user) must first register via a first user registration module 348, a patient (i.e., a second user) must first register via a second user registration module 350, and a family member (i.e., a third user) must first register via a third user registration module 352. The second user may input profile information including name, occupation, telephone number, gender, date of birth, address, third user profile information, demographic information, and the like into the second user registration module 350 through an add new patient registration screen 336c (See FIG. 12). In the present embodiment, the second user listing 336a includes a button 336b configured to generate and display an add new second user screen 336c. Once registered, the user may then access the system 100 through the portal 300 by inputting their login credentials, which correspond with the login credentials previously registered and stored within the system 100 by using a login screen 330a generated and displayed by the login module 330.

For purposes of illustration only, the first user (i.e., the health care provider or clinician) will be used to describe the various aspects of the interactive scheduling web portal system 100 according to an exemplary embodiment of the present general inventive concept. However, the present general inventive concept is not limited thereto.

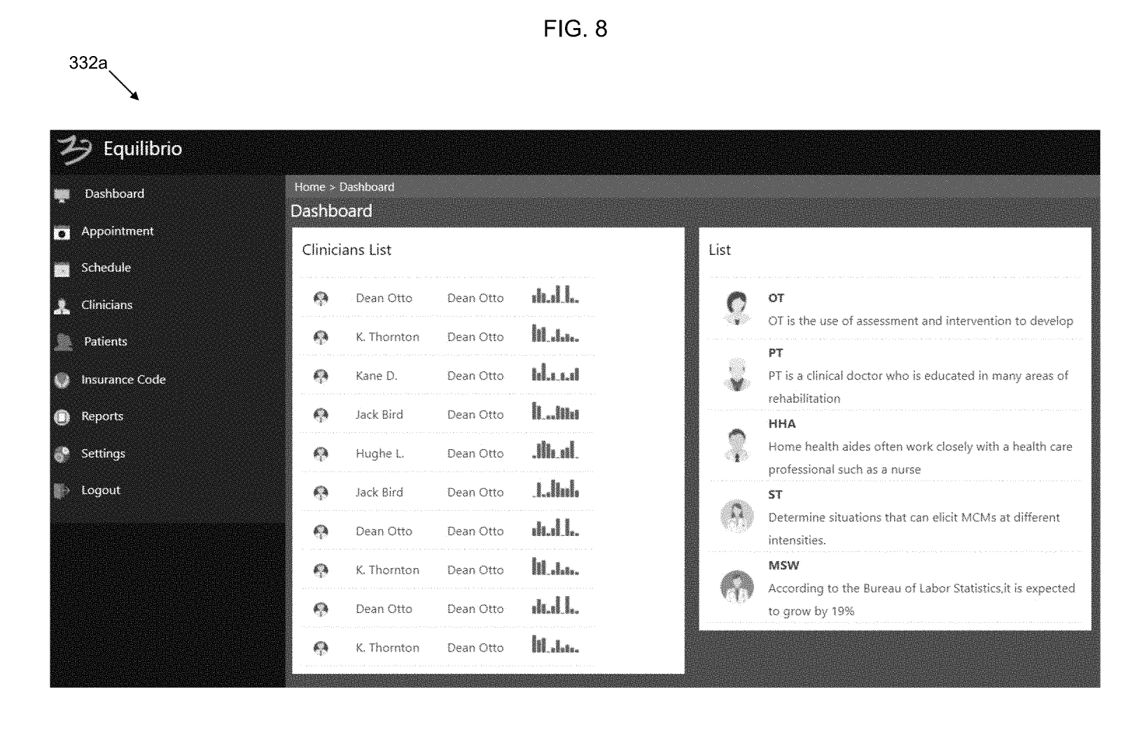

Referring now to FIG. 8, upon authentication of a user (e.g., an administrator), the system 100 generates and outputs to a display screen 24 an interactive dashboard user interface 332a using a dashboard module 332 which provides the administrator user with access to a first user list module 334 (i.e., “Clinicians”), a second user list module 336 (i.e., “Patients”), a third user list module 338, an appointment module 340 (i.e., “Appointment”), and a schedule module 342 (i.e., “Schedule”). The system 100 may only generate and display information corresponding to a particular first user to that first user. For instance, for a first user named Kane D., the system 100 would only generate and display patients, schedules, and appointments assigned to Kane D. In the present embodiment, only an administrator user may have access to an interactive dashboard user interface 332a which includes all of the registered clinicians and patients' schedules and appointments stored in the system 100. In addition, the system 100 would only allow the administrator user to filter/sort/view/edit/override all schedules of clinicians/patients registered with the system 100. In alternative embodiments, the administrator user may further input date of hire information, first user biography information, first user photo, and general interest and hobby information for each first user within the system 100. However, the present general inventive concept is not limited thereto.

The dashboard module 332 may be designed and/or configured to generate and display the interactive dashboard user interface 332a illustrating each first user's workload or amount of appointments scheduled for each day, in real-time. This data may be represented as a bar chart over a period of seven days. However, the present general inventive concept is not limited thereto. That is, in alternative embodiments, the dashboard module 332 may generate and display a heat map using a specific color to indicate a greater amount of appointments on that day.

The first user list module 334 may be designed and/or configured to generate and display a first user listing 334a of all first users (i.e., clinicians) registered in the system 100 from the dashboard user interface 332a. The first user listing 334a includes all of the first users profile information including name, contact information, photo identification, title, practice area, address, ID number, and the like inputted via the first user registration module 348 and/or the administrator. The first user listing 334a may further include a button 334b configured to generate and display an add new first user screen 334c. However, clinicians (i.e., first users) may only access their schedules and view blocked-off times for other practice areas/disciplines or days/hours the patients (i.e., second users) are unavailable.

The second user list module 336 may be designed and/or configured to generate and display a second user listing 336a of all second users (i.e., patients) registered in the system 100 from the dashboard user interface 332a. The second user listing 336a includes all of the second users profile information including name, occupation, telephone number, gender, date of birth, address, third user profile information, demographic information, and the like. In the present embodiment, the second user listing 336a includes a button 336b configured to generate and display an add new second user (i.e., patient) screen 336c. In the present embodiment, the third user may correspond to a family member of the second user. However, the present general inventive concept is not limited thereto.

The appointment module 340 may be designed and/or configured to generate and display an appointment overview screen 340a. The appointment overview screen 340a includes all appointments scheduled for all first users registered within the system 100, only accessible by the administrator. That is, in the present exemplary embodiment, the appointment overview screen 340a will only display appointments scheduled for the authenticated first user. The appointment module 340 further generates a visual display of all the scheduled appointments for each first user registered within the system 100. The appointment overview screen 340a may further include a button 340b to add a new appointment to the calendar and remove an existing appointment via a schedule appointment screen 340c.

The schedule module 342 may be designed and/or configured to generate and display a schedule overview screen 340a. The schedule overview screen 340a includes all appointments scheduled for all first users registered within the system 100, only accessible by the administrator. That is, in the present exemplary embodiment, the schedule overview screen 340a will only display appointments scheduled for the authenticated first user. The schedule overview screen 340a includes a visual display of all the scheduled appointments for each first user registered within the system 100. The schedule overview screen 340a may further include a button 340b configured to request the schedule module 342 to generate and display a new appointment screen 340c to input and schedule a new appointment.

The present general inventive concept also provides an interactive scheduling web portal application or program that allows patients to access their schedules for clinical visits by health care providers and provides an arrival window (e.g., between 2 to 4 hours) which allows patients and third parties to better prepare for care and planning. The system according to the present general inventive concept would display live updates and allow the health care provider to notify the patient in real-time of any actual or expected delays. The system may prevent fraud in the Home Health care field by confirming whether the health care provider attended the scheduled visit using GPS or various other location determining methods.

The present general inventive concept provides an interactive scheduling web portal including a plurality of executable instructions that increase an efficiency of scheduling home health care visits or appointments of health care providers with patients by allowing health care providers, patients, and other third parties (i.e., family of patients) to view real-time updates on arrival time of the health care provider to the predefined appointment location. For instance, the efficiency may be calculated based on the amount of time spent by the health care provider on each appointment divided by an amount of benefit or improvement in health of the patient. However, the present general inventive concept is not limited thereto.

The present general inventive concept also provides an interactive scheduling web portal that utilizes patient scheduling data that is translated within the program and made accessible to health care providers, patients, and third parties to check whether or not the health care provider will be arriving at the appointment location on time.

The present general inventive concept also provides an interactive scheduling web portal that allows patients and other third parties (i.e., family of patients) to access live pictures of the patients to visualize progress of treatment.

The interactive scheduling web portal according to the present general inventive concept may further allow referral sources to record snap-shots of progress related to centers for medical services (CMS) guidelines.

The present general inventive concept also provides an interactive scheduling web portal application or program that measures, tracks, and records data to evaluate efficiency and/or productivity for each health care provider.

The present general inventive concept also provides an interactive scheduling web portal application or program that may be stored on a computer readable medium and executed using a processor or computer. However, the present general inventive concept may not be limited thereto. That is, in alternative exemplary embodiments, the secure communication web portal application of the present inventive concept may be stored and executed on a remote server and accessed using a mobile device or computer.

In exemplary embodiments, the interactive scheduling web portal system and method further allows the health care provider to simply select one button to instantly notify the patient that the health care provider will be 15 minutes, 30 minutes, or 1 hour late to the scheduled appointment.

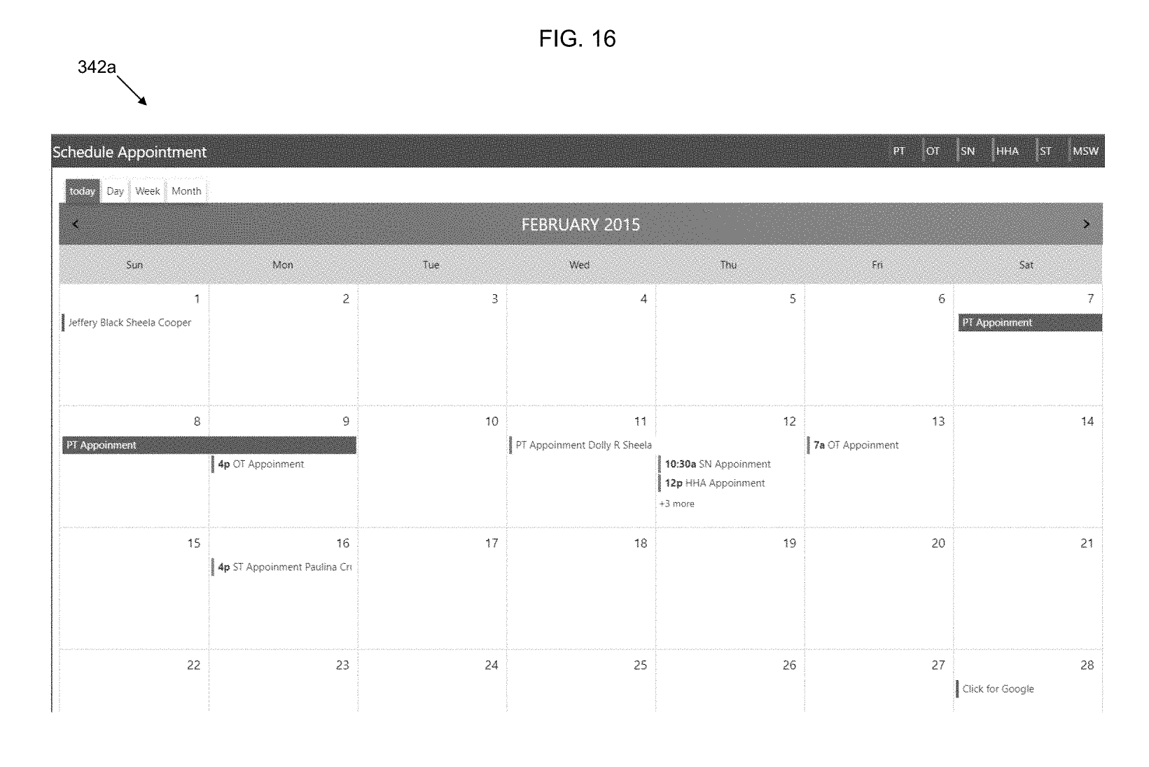

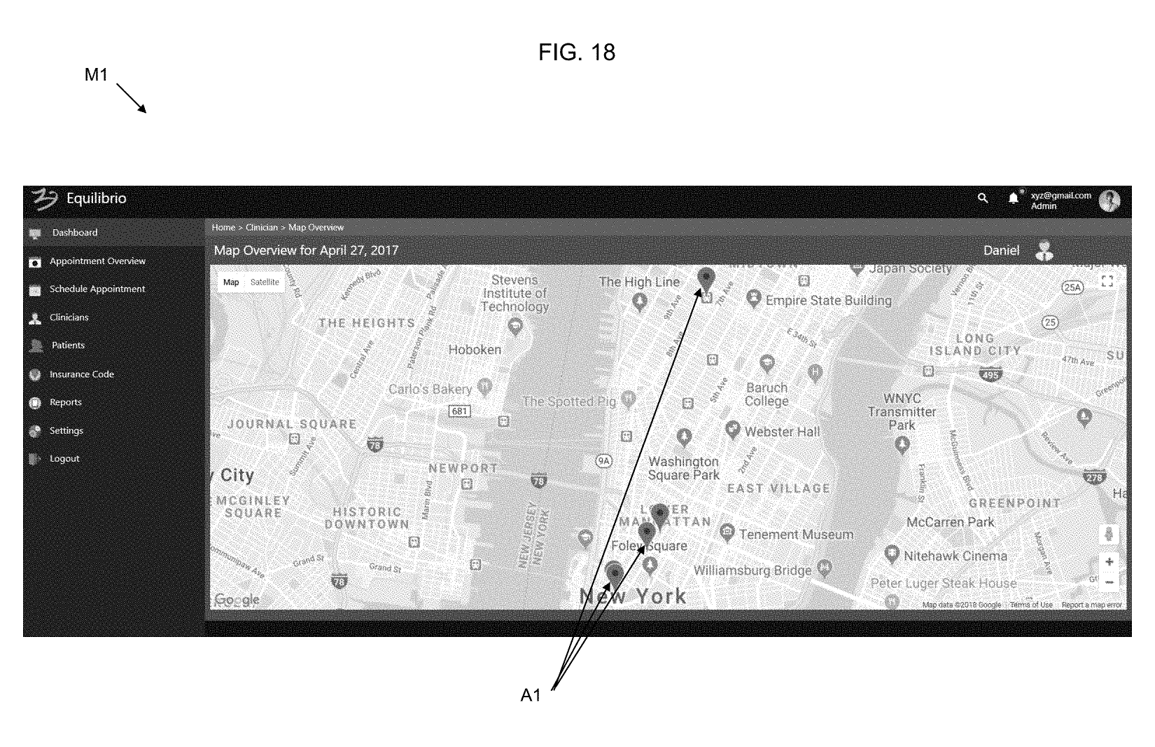

FIG. 16 illustrates a screen shot displaying a schedule produced by the scheduling portal. This schedule may be rendered on patient, patient family member or on the medical provider mobile devices. The first user may visually see all of his or her appointments scheduled for a particular day, week, or month. The first user may then select a single button to display a map and directions to each scheduled appointment, without exiting the main screen of the instructions. In addition, the first user may select a single button to indicate that the appointment has been completed from the same screen. The instructions may display a map overview for each selected date as shown in FIG. 18, so that the medical provider may visually see all of his or her appointments scheduled for that day on a single map. The instructions may also allow the medical provider user to select between a plurality of buttons (15 minutes, 30 minutes, and 1 hour), to notify the patient and/or administrator if the medical provider is running late to each appointment.

The instructions 200 may allow the second and/or third user to request an appointment. The fourth user (e.g., health care administrator) may approve or deny the requested appointment. The instructions 200 will be able to track various performance metrics for each first users (i.e., health care providers). However, the present general inventive concept is not limited thereto.

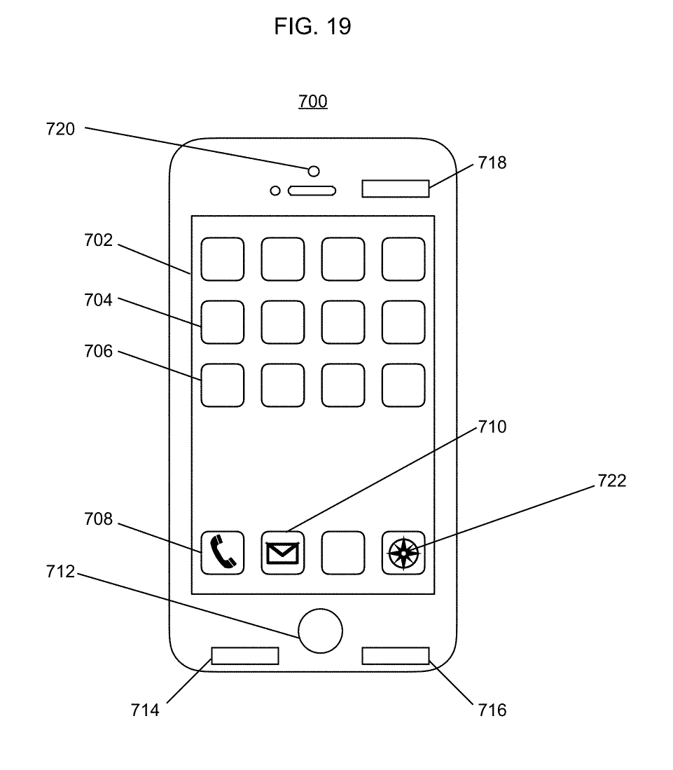

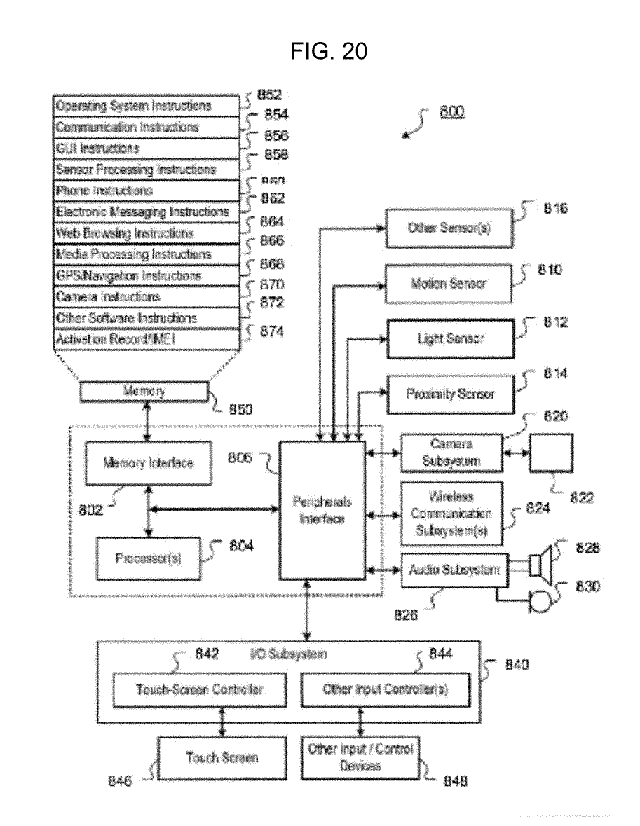

FIG. 19 illustrates a block diagram of an example portable computing device implementing the interactive scheduling web portal system, according to the present general inventive concept. FIG. 20 illustrates a block diagram of another example embodiment of a portable computing device used to implement the interactive scheduling web portal system, according to the present general inventive concept.

FIG. 19 illustrates a block diagram of an exemplary mobile device on which the invention can be implemented. The mobile device 700 can be, for example, a personal digital assistant, a cellular telephone, a network appliance, a camera, a smart phone, an enhanced general packet radio service (EGPRS) mobile phone, a network base station, a media player, a navigation device, an email device, a game console, or a combination of any two or more of these data processing devices or other data processing devices.

In some implementations, the mobile device 700 includes a touch-sensitive display 702. The touch-sensitive display 702 may implement liquid crystal display (LCD) technology, light emitting polymer display (LPD) technology, or some other display technology. The touch-sensitive display 702 can be sensitive to haptic and/or tactile contact with a user.

In some implementations, the touch-sensitive display 702 can comprise a multi-touch-sensitive display 702. A multi-touch-sensitive display 702 can, for example, process multiple simultaneous touch points, including processing data related to the pressure, degree and/or position of each touch point. Such processing facilitates gestures and interactions with multiple fingers, chording, and other interactions. Other touch-sensitive display technologies can also be used, e.g., a display in which contact is made using a stylus or other pointing device.

In some implementations, the mobile device 700 can display one or more graphical user interfaces on the touch-sensitive display 702 for providing the user access to various system objects and for conveying information to the user. In some implementations, the graphical user interface can include one or more display objects 704, 706. In the example shown, the display objects 704, 706, are graphic representations of system objects. Some examples of system objects include device functions, applications, windows, files, alerts, events, or other identifiable system objects. However, the present general inventive concept is not limited thereto.

In some implementations, the mobile device 700 can implement multiple device functionalities, such as a telephony device, as indicated by a phone object 708; an e-mail device, as indicated by the e-mail object 710; a network data communication device; a Wi-Fi base station device (not shown); and a media processing device. In some implementations, device functionalities can be accessed from a top-level graphical user interface, such as the graphical user interface illustrated in the figure. Touching one of the objects 704, 706, 708, or 710 can, for example, invoke corresponding functionality.

In some implementations, the mobile device 700 can implement network distribution functionality. For example, the functionality can enable the user to take the mobile device 700 and its associated network while traveling. In particular, the mobile device 700 can extend Internet access (e.g., Wi-Fi) to other wireless devices in the vicinity. For example, mobile device 700 can be configured as a base station for one or more devices. As such, mobile device 700 can grant or deny network access to other wireless devices.

In some implementations, upon invocation of device functionality, the graphical user interface of the mobile device 700 changes or is augmented or replaced with another user interface or user interface elements, to facilitate user access to particular functions associated with the corresponding device functionality. For example, in response to a user touching the phone object 708, the graphical user interface of the touch-sensitive display 702 may present display objects related to various phone functions; likewise, touching of the email object 708 may cause the graphical user interface to present display objects related to various e-mail functions; touching the Web object may cause the graphical user interface to present display objects related to various Web-surfing functions; and touching the media player object 94 may cause the graphical user interface to present display objects related to various media processing functions.

In some implementations, the top-level graphical user interface environment or state can be restored by pressing a button 712 located near the bottom of the mobile device 700. In some implementations, each corresponding device functionality may have corresponding “home” display objects displayed on the touch-sensitive display 702, and the graphical user interface environment can be restored by pressing the “home” display object.

In some implementations, the top-level graphical user interface can include additional display objects, such as a short messaging service (SMS) object, a calendar object, a photos object, a camera object, a calculator object, a stocks object, a weather object, a maps object, a notes object, a clock object, an address book object, a settings object, and an app store object. Touching the SMS display object can, for example, invoke an SMS messaging environment and supporting functionality; likewise, each selection of a display object can invoke a corresponding object environment and functionality.

Additional and/or different display objects can also be displayed in the graphical user interface. For example, if the device 700 is functioning as a base station for other devices, one or more “connection” objects may appear in the graphical user interface to indicate the connection. In some implementations, the display objects can be configured by a user, e.g., a user may specify which display objects are displayed, and/or may download additional applications or other software that provides other functionalities and corresponding display objects.

In some implementations, the mobile device 700 can include one or more input/output (I/O) devices and/or sensor devices. For example, a speaker 714 and a microphone 716 can be included to facilitate voice-enabled functionalities, such as phone and voice mail functions. In some implementations, an up/down button for volume control of the speaker 714 and the microphone 716 can be included. In some implementations, a loud speaker 714 can be included to facilitate hands-free voice functionalities, such as speaker phone functions.

In some implementations, a proximity sensor 718 can be included to facilitate the detection of the user positioning the mobile device 700 proximate to the user's ear and, in response, to disengage the touch-sensitive display 702 to prevent accidental function invocations.

Other sensors can also be used. For example, in some implementations, an ambient light sensor can be utilized to facilitate adjusting the brightness of the touch-sensitive display 702. In some implementations, an accelerometer can be utilized to detect movement of the mobile device 700, as indicated by the directional arrows. Accordingly, display objects and/or media can be presented according to a detected orientation, e.g., portrait or landscape. In some implementations, the mobile device 700 may include circuitry and sensors for supporting a location determining capability, such as that provided by the global positioning system 722 (GPS) or other positioning systems (e.g., systems using Wi-Fi access points, television signals, cellular grids, Uniform Resource Locators (URLs)). In some implementations, a positioning system (e.g., a GPS receiver) can be integrated into the mobile device 700 or provided as a separate device that can be coupled to the mobile device 700 through an interface to provide access to location-based services.

The mobile device 700 can also include a camera lens and sensor 720. In some implementations, the camera lens and sensor 720 can be located on the back surface of the mobile device 700. The camera can capture still images and/or video.

The mobile device 700 can also include one or more wireless communication subsystems, such as an 802.11b/g communication device, and/or a BLUETOOTH communication device. Other communication protocols can also be supported, including other 802.x communication protocols (e.g., WiMax, Wi-Fi, 3G, LTE), code division multiple access (CDMA), global system for mobile communications (GSM), Enhanced Data GSM Environment (EDGE), etc.

In some implementations, the port device, e.g., a Universal Serial Bus (USB) port, or a docking port, or some other wired port connection, is included. The port device can, for example, be utilized to establish a wired connection to other computing devices, such as other communication devices 700, network access devices, a personal computer, a printer, or other processing devices capable of receiving and/or transmitting data. In some implementations, the port device allows the mobile device 700 to synchronize with a host device using one or more protocols, such as, for example, the TCP/IP, HTTP, UDP and any other known protocol. In some implementations, a TCP/IP over USB protocol can be used.

FIG. 8 is a block diagram 800 of an example implementation of the mobile device 700. The mobile device 700 can include a memory interface 802, one or more data processors, image processors and/or central processing units 804, and a peripherals interface 806. The memory interface 802, the one or more processors 804 and/or the peripherals interface 806 can be separate components or can be integrated in one or more integrated circuits. The various components in the mobile device 700 can be coupled by one or more communication buses or signal lines.

Sensors, devices and subsystems can be coupled to the peripherals interface 806 to facilitate multiple functionalities. For example, a motion sensor 810, a light sensor 812, and a proximity sensor 814 can be coupled to the peripherals interface 806 to facilitate the orientation, lighting and proximity functions described above. Other sensors 816 can also be connected to the peripherals interface 806, such as a positioning system (e.g., GPS receiver), a temperature sensor, a biometric sensor, or other sensing device, to facilitate related functionalities.

A camera subsystem 820 and an optical sensor 822, e.g., a charged coupled device (CCD) or a complementary metal-oxide semiconductor (CMOS) optical sensor, can be utilized to facilitate camera functions, such as recording photographs and video clips.

Communication functions can be facilitated through one or more wireless communication subsystems 824, which can include radio frequency receivers and transmitters and/or optical (e.g., infrared) receivers and transmitters. The specific design and implementation of the communication subsystem 824 can depend on the communication network(s) over which the mobile device 700 is intended to operate. For example, a mobile device 700 may include communication subsystems 824 designed to operate over a GSM network, a GPRS network, an EDGE network, a Wi-Fi or WiMax network, and a BLUETOOTH network. In particular, the wireless communication subsystems 2224 may include hosting protocols such that the device 700 may be configured as a base station for other wireless devices.

An audio subsystem 826 can be coupled to a speaker 828 and a microphone 830 to facilitate voice-enabled functions, such as voice recognition, voice replication, digital recording, and telephony functions. The I/O subsystem 840 can include a touch screen controller 842 and/or other input controller(s) 844. The touch-screen controller 842 can be coupled to a touch screen 846. The touch screen 846 and touch screen controller 842 can, for example, detect contact and movement or break thereof using any of multiple touch sensitivity technologies, including but not limited to capacitive, resistive, infrared, and surface acoustic wave technologies, as well as other proximity sensor arrays or other elements for determining one or more points of contact with the touch screen 846.

The other input controller(s) 844 can be coupled to other input/control devices 848, such as one or more buttons, rocker switches, thumb-wheel, infrared port, USB port, and/or a pointer device such as a stylus. The one or more buttons (not shown) can include an up/down button for volume control of the speaker 828 and/or the microphone 830.

In one implementation, a pressing of the button for a first duration may disengage a lock of the touch screen 846; and a pressing of the button for a second duration that is longer than the first duration may turn power to the mobile device 700 on or off. The user may be able to customize a functionality of one or more of the buttons. The touch screen 846 can, for example, also be used to implement virtual or soft buttons and/or a keyboard.

In some implementations, the mobile device 700 can present recorded audio and/or video files, such as MP3, AAC, and MPEG files. In some implementations, the mobile device 700 can include the functionality of an MP3 player. The mobile device 700 may, therefore, include a 32-pin connector that is compatible with the MP3 player. Other input/output and control devices can also be used.

The memory interface 802 can be coupled to memory 850. The memory 850 can include high-speed random-access memory and/or non-volatile memory, such as one or more magnetic disk storage devices, one or more optical storage devices, and/or flash memory (e.g., NAND, NOR). The memory 850 can store an operating system 852, such as Darwin, RTXC, LINUX, UNIX, OS X, ANDROID, IOS, WINDOWS, or an embedded operating system such as VxWorks. The operating system 852 may include instructions for handling basic system services and for performing hardware dependent tasks. In some implementations, the operating system 852 can be a kernel (e.g., UNIX kernel).

The memory 850 may also store communication instructions 854 to facilitate communicating with one or more additional devices, one or more computers and/or one or more servers. The memory 850 may include graphical user interface instructions 856 to facilitate graphic user interface processing including presentation, navigation, and selection within an application store; sensor processing instructions 858 to facilitate sensor-related processing and functions; phone instructions 860 to facilitate phone-related processes and functions; electronic messaging instructions 862 to facilitate electronic-messaging related processes and functions; web browsing instructions 864 to facilitate web browsing-related processes and functions; media processing instructions 866 to facilitate media processing-related processes and functions; GPS/Navigation instructions 868 to facilitate GPS and navigation-related processes and instructions; camera instructions 870 to facilitate camera-related processes and functions; and/or other software instructions 872 to facilitate other processes and functions.

Each of the above identified instructions and applications can correspond to a set of instructions for performing one or more functions described above. These instructions need not be implemented as separate software programs, procedures or modules. The memory 850 can include additional instructions or fewer instructions. Furthermore, various functions of the mobile device 700 may be implemented in hardware and/or in software, including in one or more signal processing and/or application specific integrated circuits.

The exemplary general inventive concept also provides an interactive scheduling web portal that includes a virtual home exercise plan application.

This virtual home exercise plan application may be housed within the main scheduling Application and will be able to help the patient and family look at “live pictures” of the patient themselves performing a “Home Exercises” using a snap-shot format with proper positioning and use of equipment if needed. This format will have the capability to insert numbers for repetitions of exercise and sets to be done daily or multiple times a day.

This virtual home exercise plan application may allow patients and their families to access these custom-made home exercise programs using the “actual picture” taken of the patient doing them with best form and set up possible using a SNAP SHOT format from a cellular tablet. These pictures can be entered onto a pre-configured template for easy viewing and readability for the patient and family to understand how the patient should be performing each particular exercise photographed by the therapist/nurse using their tablet or device.

This virtual home exercise plan application will allow patients and their families to easily access these Home Exercise pictures as the most interactive way of learning correct ways to perform the exercise and having pictures of the patient to refer to, creating more compliance with doing the exercises properly. This concept will allow patients and families members to track what they are doing with “actual pictures of them” performing it to the best of their ability further adding visual proof of progress and ability to perform in Home Health Care.

The virtual home exercise plan application will include a live snap shot application using pictures or videos if needed and will help create an intimate Clinician to Client/Patient relationship with their care, providing the most up to date and interactive planning, charting, updates on their progress.

The virtual home exercise plan application can also be used by physicians and other referral sources involved in the patient care to better convey progress with “live Pictures” of the patient that can be updated in the patient EMR or hard paper chart, if needed.

In an exemplary embodiment of the present general inventive concept, the interactive scheduling web portal application or program AND the virtual home exercise plan application may operate as follows: First, a clinician or other health care provider uses a tablet or smart phone for Home Health purposes only. The clinician positions and prepares patient with how to properly perform exercise thru practice and family education.

Once the patient is able to perform the exercise to best of his or her ability, the clinician can then photograph each patient performing a repetition of the exercise with proper positioning and equipment set up needed in the home to perform with the family. The clinician may then upload the pictures to a pre-developed application interface using ANDROID™ and APPLE™ technology in a predetermined lay out to allow the patient and/or the patient's family to view and understand how to perform exercise, why, and important clues along with a number of reps, sets, times of day to perform (frequency), as well as the necessary equipment for each exercise.

The template can then be automatically emailed to the patient e-mail or approved family member e-mail address to access template for printing or to refer. Also, this template and home exercise plan pictures can be accessed with patient or family using the interactive system by signing up using their identification number and password for the interactive system through the web or mobile application.

In an exemplary embodiment, the following is a listing of the components needed in the order of operation. However, the present general inventive concept is not limited thereto. For instance, the present invention includes a Smart Phone, tablet or other device, an image capturing application stored and executed on this device. The device may be linked to snap shot application or is able to upload captured pictures to this application either via web-based or mobile versions. The pictures are then stored on this device and converted to application format and are then entered onto a template which allows the clinician to easily organize and enter additional notes or instructions regarding each exercise to be viewed by the patient or family member. This may include name of exercise, equipment necessary to perform exercise, the amount of repetitions and sets, and the frequency the patient should perform each exercise.

The captured images of the exercises performed by the patient are saved through the application executed on the device and may be transmitted to a server through an internet connection. The exemplary embodiment allows various connectivity options for email delivery to patient/family or patient/family signed up to use application through a central web console in order to access exercise plan pictures and info, and also allows the ability to communicate through “chats” with clinicians or Home Health office regarding exercise plan or questions, in real-time.

In exemplary embodiments, the present general inventive concept also provides an interactive scheduling web portal application or program that provides access to calendars and the Pecos directory along with patient data and orders within the system.

In exemplary embodiments, the present general inventive concept also provides an interactive scheduling web portal application or program that provides allows users (e.g., patients or health care providers) to upload images or videos to a secure database to track a health progress of the patient and also to allow the patient to see which health care provider will be coming to their home for security purposes.

In exemplary embodiments, the present general inventive concept also provides an interactive scheduling web portal application or program that collects data regarding efficiency of the appointment, efficiency of the home health care provider, the time required for each visit or appointment, as well as a success rate of the agency and adherence to Medicare guidelines. The system may further include a feedback loop which collects and analyzes previously collected data to suggest future routes to the patient or treatment plans that have been successful.

The present general inventive concept provides an interactive scheduling web portal system accessible from a smart phone, tablet, or other mobile device. The interactive scheduling web portal system and method allows a health care provider to quickly visualize all of his or her scheduled appointments with patients on a single screen and allows the health care provider to press a single button to display a map and directions to the appointment location (e.g., patient's home). In addition, the interactive scheduling web portal system and method allows the health care provider to press a single button to record within a central database that the appointment has been completed.

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A scheduling portal comprising:
a processor;
a memory on which are stored machine readable instructions that when executed by the processor, cause the processor to:
access medical provider data for a plurality of medical providers and store the medical provider data in a database;
receive patient parameters from a patient mobile device and store the patient parameters in the database;
determine if the patient parameters contain a scheduling request for at least one of the medical providers having the medical provider data stored in the database;
schedule a requested appointment for the patient with the medical provider if the patient parameters contain a scheduling request;
update a medical provider calendar in the database; and
send the updated medical provider calendar to a medical provider mobile device.

2. The scheduling portal of claim 1, wherein the instructions are further to cause the processor to check medical provider availability for the requested appointment.

3. The scheduling portal of claim 2, wherein the instructions are further to cause the processor to send a notification to the patient mobile device if the medical provider is not available.

4. The scheduling portal of claim 1, wherein the instructions are further to cause the processor to send a notification to the patient mobile device if the medical provider is available, wherein the notification renders the updated medical provider calendar on the patient mobile device.

5. The scheduling portal of claim 1, wherein the instructions are further to cause the processor to track medical provider movement and update the medical provider calendar.

6. The scheduling portal of claim 5, wherein the instructions are further to cause the processor to send the updated medical provider calendar to the patient mobile device to render the updated medical provider calendar to the patient.

7. The scheduling portal of claim 6, wherein the instructions are further to cause the processor to send and render a map depicting locations of appointments to the medical provider mobile device.

8. The scheduling portal of claim 1, wherein the database is a local database.

9. The scheduling portal of claim 1, wherein the database is a cloud storage.

10. A method for scheduling appointments using a scheduling portal, the method comprising:
accessing, by a processor of the scheduling portal, medical provider data for a plurality of medical providers and store the medical provider data in a database;
receiving, by the processor of the scheduling portal, patient parameters from a patient mobile device and storing the patient parameters in the database;
determining, by the processor of the scheduling portal, if the patient parameters contain a scheduling request for at least one of the medical providers having the medical provider data stored in the database;
scheduling, by the processor of the scheduling portal, a requested appointment for the patient with the medical provider if the patient parameters contain a scheduling request;
updating, by the processor of the scheduling portal, a medical provider calendar in the database; and
sending, by the processor of the scheduling portal, the updated medical provider calendar to a medical provider mobile device.

11. The method of claim 10, further comprising checking medical provider availability for the requested appointment.

12. The method of claim 11, further comprising sending a notification to the patient mobile device if the medical provider is available, wherein the notification renders the updated medical provider calendar on the patient mobile device.

13. The method of claim 10, further comprising tracking medical provider movement and updating the medical provider calendar based on a GPS location retrieved from the medical provider mobile device.

14. The method of claim 13, further comprising sending the updated medical provider calendar to the patient mobile device to render the updated medical provider calendar to the patient.

15. The method of claim 14, further comprising sending a map depicting locations of appointments to the medical provider mobile device to be rendered to the medical provider.

16. An automated scheduling portal comprising:
a processor;
a memory on which are stored machine readable instructions that when executed by the processor, cause the processor to:
receive patient parameters from a patient mobile device and store the patient parameters in a database;
retrieve pre-stored medical provider data from the database based on the patient parameters;
send the medical provider data to the patient mobile device;
receive a scheduling request for the medical provider from the patient mobile device;
schedule an appointment for the patient with the medical provider;
update a medical provider calendar in the database; and
send the updated medical provider calendar to a medical provider mobile device.

17. The automated scheduling portal of claim 16, wherein the instructions are further to cause the processor to derive a patient family member mobile device data from the patient parameters.

18. The automated scheduling portal of claim 17, wherein the instructions are further to cause the processor to provide the appointment data to the patient family member mobile device.

19. The automated scheduling portal of claim 16, wherein the instructions are further to cause the processor to track medical provider movement and update the medical provider calendar based on a GPS location retrieved from the medical provider mobile device.

20. The automated scheduling portal of claim 19, wherein the instructions are further to cause the processor to send a map depicting a location of medical provider appointments to the patient family member mobile device to be rendered to the patient family member.
